(19) United States
(12) Patent Application Publication
(10) Pub. No.: US 2014/0100401 A1
(43) Pub. Date: Apr. 10, 2014

(54) CATALYST FOR THE ALKYLATION OF AROMATIC HYDROCARBONS

(71) Applicant: Saudi Basic Industries Corporation, Riyadh (SA)

(72) Inventor: Debasish Das, Manjusar (IN)

(73) Assignee: Saudi Basic Industries Corporation, Riyadh (SA)

(21) Appl. No.: 14/116,229

(22) PCT Filed: May 3, 2012
(86) PCT No.: PCT/EP2012/001889

§ 371 Date: Nov. 15, 2013

(30) Foreign Application Priority Data
May 9, 2011 (EP) 11003778.5

(51) Int. Cl. B01J 29/70; C07C 2/66; C07C 2/68

(52) U.S. Cl. CPC B01J 29/7007; C07C 2/68; C07C 2/66
USPC 585/462; 502/64; 585/446

(57) ABSTRACT

The present invention relates to catalyst composition prepared by a method wherein an aluminosilicate zeolite having its pores filled with templating agent with a specific organic silicon compound to deposit said organic silicon compound on the surface of the zeolite to provide an organosilicon treated catalyst precursor; and calcining the organosilicon treated catalyst precursor under conditions sufficient to remove the templating agent from the zeolite. Furthermore, the present invention relates to a method for preparing said catalyst composition and a process for alkylation of an aromatic hydrocarbon comprising contacting the catalyst composition of the present invention with a feed stream comprising said aromatic hydrocarbon and an alkylating agent under aromatic alkylation conditions.

### CROSS REFERENCE TO RELATED APPLICATION

The present application is a 371 application of PCT/EP2012/001889, filed May 3, 2012, which claims priority to European Application No. 11003778.5, filed May 9, 2011, the contents of which are incorporated by reference in their entirety.

The present invention relates to catalyst composition prepared by a method wherein an aluminosilicate zeolite having its pores filled with templating agent with a specific organic silicon compound to deposit said organic silicon compound on the surface of the zeolite to provide an organosilicon treated catalyst precursor; and calcining the organosilicon treated catalyst precursor under conditions sufficient to remove the templating agent from the zeolite. Furthermore, the present invention relates to a method for preparing said catalyst composition and a process for alkylation of an aromatic hydrocarbon comprising contacting the catalyst composition of the present invention with a feed stream comprising said aromatic hydrocarbon and an alkylating agent under aromatic alkylation conditions.

It has been previously described that calcined surface-modified zeolite catalysts are useful in aromatic alkylation processes. For instance, U.S. Pat. No. 5,723,710 describes a process for preparing cumene by the alkylation of benzene with propylene using a zeolite catalyst obtained by treating templated zeolite beta with a low concentration of a strong mineral acid followed by calcination. It is taught in U.S. Pat. No. 5,723,710 that the therein described catalyst has an improved resistance against catalyst deactivation under normal process conditions.

U.S. Pat. No. 5,689,025 describes a process for ethylbenzene production that involves contacting a hydrocarbon feedstream including benzene and ethylene, under alkylation conditions, with a catalytic molecular sieve which has been modified by being ex-situ selectivated with a silicon compound. The ex-situ selectivation involves exposing the molecular sieve to at least two selectivation sequences, each selectivation sequence comprising contacting the catalyst with a silicon compound followed by calcination. It is taught that the selectivated molecular sieve catalyst has an improved shape-selectivity for ethyl benzene over xylenes in a process for the alkylation of benzene with ethylene.

A major drawback of conventional zeolite-based aromatic alkylation catalyst is that they quickly become deactivated by impurities that are commonly comprised in the aromatic feed. The purity requirements for the aromatic feedstream in an aromatic alkylation processes accordingly are very strict. For instance, the maximum acceptable content of sulfur impurities in the feed of a conventional process for benzene alkylation must be less than 1 ppm. Other impurities, such as olefinic hydrocarbons also are known to have an adverse effect on process stability. Commonly, the bromine index of the feed of a conventional process for benzene alkylation must be less than 10.

It was an object of the present invention to provide a benzene alkylation catalyst that has an improved resistance to feed impurities.

The solution to the above problem is achieved by providing the embodiments as described herein below and as characterized in the claims. Accordingly, the present invention provides a catalyst composition obtainable by the method for preparing a catalyst composition comprising the steps of:

(a) contacting an aluminosilicate zeolite having its pores filled with templating agent with an organic silicon compound to deposit said organic silicon compound on the surface of the zeolite to provide an organosilicon treated catalyst precursor; and

(b) calcining the organosilicon treated catalyst precursor under conditions sufficient to remove the templating agent from the zeolite,

wherein the organic silicon compound is selected from the group consisting of alkyldisilazane, alkylalkoxysilane and haloalkylsilane.

The organic silicon compound used in the method for preparing a catalyst composition of the present invention is selected from the group consisting of:

(I) alkyldisilazane having the general formula

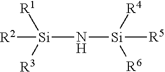

wherein R¹, R², R³, R⁴, R⁵ and R⁶ are alkyl groups independently selected from the group consisting of methyl; ethyl; propyl; butyl; pentyl; hexyl; heptyl; octyl; nonyl; and decyl;

(II) alkylalkoxysilane having the general formula, either of:

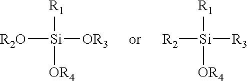

wherein R₁, R₂, R₃ and R₄ are alkyl groups independently selected from the group consisting of methyl; ethyl; propyl; and butyl; and

(III) haloalkylsilane having the general formula

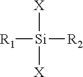

wherein R₁ and R₂ are alkyl groups independently selected from the group consisting of methyl; ethyl; propyl; and butyl and wherein X is an halogen element selected from the group consisting of fluorine (F), chlorine (Cl), bromine (Br) and iodine (I). Preferably, the halogen element is chlorine (Cl).

In the context of the present invention, it was surprisingly found that the zeolite-based catalyst prepared by the method of the present invention has a significantly improved resistance to impurities comprised in the aromatic feedstream when compared with zeolite-based benzene alkylation catalysts of the prior art. This has the profound advantage that an aromatic alkylation process which uses the catalyst of the present invention is much more robust against fluctuations in feedstream purity when compared to conventional aromatic alkylation catalysts. Moreover, it is now possible to routinely use less pure aromatic hydrocarbon compositions which otherwise are not suitable as a feedstream in a process for aromatic alkylation can be used without prior purification or pre-treatment for aromatic alkylation.

The catalyst composition of the present invention can be readily distinguished from known zeolite-based benzene alkylation catalyst compositions by its remarkable resistance to feed impurities. To the best of our knowledge, no zeolite-based benzene alkylation catalysts showing a comparable resistance to feed impurities have been previously described.

Preferably, the alkyl disilazane used in the present invention is selected from the group consisting of hexamethyldisilazane and hexaethyldisilazane and most preferably is hexamethyldisilazane.

The alkoxy silane used in the present invention is preferably selected from the group consisting of methoxytrimethylsilane, ethoxytrimethylsilane, propoxytrimethylsilane, methyltrimethoxysilane, methyltriethoxysilane, ethyltrimethoxysilane, ethyltriethoxysilane, propyltrimethoxysilane and propyltriethoxysilane. The halo alkyl silane used in the present invention is preferably selected from the group consisting of dichlorodimethylsilane and dichlorodiethylsilane.

In a further aspect of the present invention a method for preparing a catalyst composition is provided. Accordingly, the present invention provides a method comprising the steps of

(a) contacting an aluminosilicate zeolite having its pores filled with templating agent with an organic silicon compound to deposit said organic silicon compound on the surface of the zeolite to provide an organosilicon treated catalyst precursor; and

(b) calcining the organosilicon treated catalyst precursor under conditions sufficient to remove the templating agent from the zeolite,

wherein the organic silicon compound is selected from the group consisting of the alkyldisilazane, alkylalkoxysilane and haloalkylsilane compounds as described herein.

In the organic silicon compound deposition step (a), the aluminosilicate zeolite having its pores filled with templating agent is contacted with an organic silicon compound to deposit said organic silicon compound on the surface of the zeolite to provide an organosilicon treated catalyst precursor. It is essential for the present invention that the pores filled with templating agent when the zeolite is contacted with the organic silicon compound.

In the calcination step (b), the organosilicon treated catalyst precursor is calcined under conditions sufficient to remove the templating agent from the zeolite. The conditions used in the calcination step (b) can be readily determined by the skilled person. Preferably, the zeolite is calcined in step (b) at a temperature of 450-600° C. for 3-8 hrs in an oxygen comprising atmosphere. Preferably, the calcination step is performed in atmospheric air.

As used herein, the term “aluminosilicate zeolite” or “zeolite” relates to an aluminosilicate molecular sieve. These inorganic porous materials are well known to the skilled man. An overview of their characteristics is for example provided by the chapter on Molecular Sieves in Kirk-Othmer Encyclopedia of Chemical Technology, Volume 16, p 811-853; in Atlas of Zeolite Framework Types, 5^{th} edition, (Elsevier, 2001). Preferably, the zeolite is a large pore size aluminosilicate zeolite. Most preferably the zeolite is beta zeolite. Other suitable zeolites include, but are not limited to zeolite Y and mordenite. The term “large pore zeolite” is commonly used in the field of zeolite catalysts. Accordingly, a large pore size zeolite is a zeolite having a pore size of 6 to 15 Å. Suitable large pore size zeolites are 12-ring zeolites. i.e. the pore is formed by a ring consisting of 12 SiO₄ tetrahedra. Preferably, zeolites having constraint index (CI) 0.6-2.0 are used in the present invention. Methods for determining the CI of a given zeolite are well known in the art; see e.g. U.S. Pat. No. 4,016,218. Zeolite preferably is in the as-synthesized form. Silica (SiO₂) to alumina (Al₂O₃) molar ratio preferably is within a range of 20-150. The crystal size of the zeolite preferably is 0.2-20 μm.

Zeolites of the 10-ring structure type, like for example ZSM-5, are also referred to as medium pore sized; and those of the 8-ring structure type are called small pore size zeolites. In the above cited Atlas of Zeolite Framework Types various zeolites are listed based on ring structure.

Optionally, the zeolite is washed in organic solvent before the calcination step (b) is performed. A preferred organic solvent used in the optional washing step is toluene. It is believed that the unreacted organic silicon compound is removed from the catalyst composition during this washing step.

In a further embodiment of the present invention, a process for the alkylation of an aromatic hydrocarbon is provided comprising contacting the catalyst composition as described herein with a feed stream comprising said aromatic hydrocarbon and an alkylating agent under aromatic alkylation conditions.

Accordingly, a process for alkylation of an aromatic hydrocarbon is provided comprising preparing a catalyst composition comprising the steps of:

(a) contacting an aluminosilicate zeolite having its pores filled with templating agent with an organic silicon compound to deposit said organic silicon compound on the surface of the zeolite to provide an organosilicon treated catalyst precursor; and

(b) calcining the organosilicon treated catalyst precursor under conditions sufficient to remove the templating agent from the zeolite,

wherein the organic silicon compound is selected from the group consisting of alkyldisilazane, alkylalkoxysilane and haloalkylsilane and contacting the catalyst composition with a feed stream comprising said aromatic hydrocarbon and an alkylating agent under aromatic alkylation conditions.

As used herein, the term “feedstream impurity” is meant to describe all components comprised in the feedstream of a chemical process which adversely affect the intended chemical conversion taking place in said chemical process. It is commonly known that compounds that are commonly comprised in an aromatic feedstream, such as sulphur-comprising hydrocarbons, such as thiophene, or olefinic hydrocarbons, such as substituted alkenes including, but not limited to, methyl pentenes, methyl hexenes and cyclopentenes, have an adverse effect on an aromatic alkylation process.

Thiophene concentration in refined benzene is determined at ppm level using conventional gas chromatography with a pulse flame photometric detector (PFPD). A reproducible volume of the sample is injected in a Varian CP-3800 GC with PFPD detector and wax column for analysis. Quantitative results are obtained by the external standard technique and spiking technique using the measured peak area of thiophene. The analysis is based on the ASTM D 4735-02 standard method.

The bromine index in e.g. benzene is determined by potentiometric titration method. The Bromine index (BI) is the number of mg bromine that are bound or added by 100 g sample. The Bromine index is the fraction of reactive unsaturated compounds (mostly C═C double bonds) in the hydrocarbons encountered in the petrochemicals industry. The double bonds are split with the attachment of bromine. R—C═C—R+Br₂-→R—CBr—CBr—R. The sample is titrated with the 0.02 N Bromide-bromate solution to find out the Bromine index. The test was performed with Metrohm 798 MPT Titrino and the test method is based on the standard reference method ASTM D 2710-72 and ASTM D 5776-99.

The upper limits of BI and sulfur impurities (such as thiophene impurity) of the feed commonly acceptable in the industry are 10 and 1 ppm respectively. The BI of the feedstream used in the process of the present invention may be more than 10, preferably more than 20 and most preferably more than 25. Moreover, the feedstream used in the process of the present invention comprises may comprise more than 1 ppm of sulfur impurities, preferably more than 10 ppm of sulfur impurities, even more preferably more than 30 ppm of sulfur impurities and most preferably more than 50 ppm of sulfur impurities. Most preferably, the feedstream used in the process of the present invention has a BI of 25 or more and 50 ppm of sulfur impurities.

The terms “aromatic hydrocarbon” is very well known in the art. Accordingly, the term “aromatic hydrocarbon” relates to cyclically conjugated hydrocarbon with a stability (due to delocalization) that is significantly greater than that of a hypothetical localized structure (e.g. Kekulé structure). The most common method for determining aromaticity of a given hydrocarbon is the observation of diatropicity in the¹H NMR spectrum. Preferably, the aromatic hydrocarbon is selected from the group consisting of benzene and toluene.

The term “alkylating agent” is very well known in the art and relates to a hydrocarbon compound capable of transferring an alkyl group to the aromatic hydrocarbon. Accordingly, the alkylating agent is preferably selected from the group consisting of ethylene, propylene and linear alpha-olefins, such as 1-butene and 1-pentene.

Most preferably, the aromatic hydrocarbon is benzene and the alkylating agent is ethylene. In this case the benzene:ethylene molar ratio preferably is 3-6:1 and most preferably 4:1.

The process conditions useful in the process of the present invention, also described herein as “aromatic alkylation conditions”, can be easily determined by the person skilled in the art; see Kirk-Othmer Encyclopedia of Chemical Technology, Volume 2, p. 169-203. Accordingly, the aromatic alkylation process may be performed at a reaction temperature of 150-250° C., a pressure of 5-40 barg, and a weight hourly space velocity of 0.1-10. Preferably, the aromatic alkylation process of the present invention is performed in the liquid phase. At a process temperature of 150° C. benzene will be in liquid phase at pressure of 6 barg or more.

In the process of the present invention, the catalyst composition is preferably comprised in a fixed bed reactor or a fluidized bed reactor.

### MODE(S) FOR CARRYING OUT THE INVENTION

The present invention will now be more fully described by the following non-limiting Examples.

### EXAMPLES

### Example 1 (Comparative)

### Preparation of Unmodified Beta Zeolite Catalyst

10.0 g beta zeolite powder in ammonium form (with template) was calcined at 575° C. for 4 hours with a heating rate of 2° C./min in presence of zero air (120-150 ml/min).

### Example 2 (Comparative)

Preparation of Beta Zeolite Modified with Tetraethoxysilane

About 7.0 g dried beta zeolite powder in ammonium form was introduced into 250 ml four-neck round bottom flask equipped with addition funnel, reflux condenser, vaccum adaptor, thermometer and magnetic bar. The powder was slowly heated upto 140° C. and evacuated by vacuum up to 10 mbar while stirring. After 4 hours it was cooled down to 50° C. under stirring and then vacuum arrested.

A solution of 0.22 ml tetraethoxysilane and 50 ml anhydrous toluene was added in addition funnel. The mixture was carefully added in the RB flask at 50° C. while stirring. After addition of entire quantity, nitrogen gas was introduced into the flask and was heated slowly to reflux temperature under stirring and N₂ gas atmosphere. After 4 hours the flask was cooled down to 40° C. and nitrogen flow was stopped.

The solid was filtered and washed with anhydrous toluene and finally the filtered cake was rinsed with 30 ml of absolute ethanol and dried at 100° C. for 14 hrs. The dried material was calcined at 575° C. for 4 hours with a ramp rate of 2° C./min in presence of zero air in muffle furnace. Final yield of the dry powder was about 6.2 g.

### Example 3 (Comparative)

Preparation of Beta Zeolite Modified with Hexamethyldisiloxane

About 7.0 g dried beta zeolite powder in ammonium form was introduced into 250 ml four-neck round bottom flask equipped with addition funnel, reflux condenser, vacuum adaptor, thermometer and magnetic bar. The powder was slowly heated up to 140° C. and evacuated by vacuum up to 10 mbar while stirring. After 4 hours it was cooled down to 50° C. under stirring and then vacuum arrested.

A solution of 2.7 ml of hexamethyldisiloxane and 50 ml anhydrous toluene was added in addition funnel. The mixture was carefully added in the RB flask at 50° C. while stirring. After addition of entire quantity, nitrogen gas was introduced into the flask and was heated slowly to reflux temperature under stirring and N₂ gas atmosphere. After 4 hours the flask was cooled down to 40° C. and nitrogen flow was stopped.

The solid was filtered and washed with anhydrous toluene and finally the filtered cake was rinsed with 30 ml of absolute ethanol and dried at 100° C. for 14 hrs. The dried material was calcined at 575° C. for 4 hours with a ramp rate of 2° C./min in presence of zero air in muffle furnace. Final yield of the dry powder was about 6.2 g.

### Example 4

Preparation of Beta Zeolite Modified with Dichlorodimethylsilane

About 7.0 g dried beta zeolite powder in ammonium form was introduced into 250 ml four-neck round bottom flask equipped with addition funnel, reflux condenser, vacuum adaptor, thermometer and magnetic bar. The powder was slowly heated upto 140° C. and evacuated by vacuum up to 10 mbar while stirring. After 4 hours it was cooled down to 50° C. under stirring and then vacuum arrested.

A solution of 1.6 ml of dichlorodimethylsilane and 50 ml anhydrous toluene was added in addition funnel. The mixture was carefully added in the RB flask at 50° C. while stirring. After addition of entire quantity, nitrogen gas was introduced into the flask and was heated slowly to reflux temperature under stirring and N₂ gas atmosphere. After 4 hours the flask was cooled down to 40° C. and nitrogen flow was stopped.

The solid was filtered and washed with anhydrous toluene and finally the filtered cake was rinsed with 30 ml of absolute ethanol and dried at 100° C. for 14 hrs. The dried material was calcined at 575° C. for 4 hours with a ramp rate of 2° C./min in presence of zero air in muffle furnace. Final yield of the dry powder was about 6.3 g.

### Example 5

Preparation of Beta Zeolite Modified with Methyltrimethoxysilane

About 7.0 g dried beta zeolite powder in ammonium form was introduced into 250 ml four-neck round bottom flask equipped with addition funnel, reflux condenser, vacuum adaptor, thermometer and magnetic bar. The powder was slowly heated upto 140° C. and evacuated by vacuum up to 10 mbar while stirring. After 4 hours it was cooled down to 50° C. under stirring and then vacuum arrested.

A solution of 3.7 ml of methyltrimethoxysilane and 50 ml anhydrous toluene was added in addition funnel. The mixture was carefully added in the RB flask at 50° C. while stirring. After addition of entire quantity, nitrogen gas was introduced into the flask and was heated slowly to reflux temperature under stirring and N₂ gas atmosphere. After 4 hours the flask was cooled down to 40° C. and nitrogen flow was stopped.

The solid was filtered and washed with anhydrous toluene and finally the filtered cake was rinsed with 30 ml of absolute ethanol and dried at 100° C. for 14 hrs. The dried material was calcined at 575° C. for 4 hours with a ramp rate of 2° C./min in presence of zero air in muffle furnace. Final yield of the dry powder was about 6.1 g.

### Example 6

Preparation of Beta Zeolite Modified with Ethoxytrimethylsilane

About 7.0 g dried beta zeolite powder in ammonium form was introduced into 250 ml four-neck round bottom flask equipped with addition funnel, reflux condenser, vacuum adaptor, thermometer and magnetic bar. The powder was slowly heated upto 140° C. and evacuated by vacuum up to 10 mbar while stirring. After 4 hours it was cooled down to 50° C. under stirring and then vacuum arrested.

A solution of 4.0 ml of ethoxytrimethylsilane and 50 ml anhydrous toluene was added in addition funnel. The mixture was carefully added in the RB flask at 50° C. while stirring. After addition of entire quantity, nitrogen gas was introduced into the flask and was heated slowly to reflux temperature under stirring and N₂ gas atmosphere. After 4 hours the flask was cooled down to 40° C. and nitrogen flow was stopped.

The solid was filtered and washed with anhydrous toluene and finally the filtered cake was rinsed with 30 ml of absolute ethanol and dried at 100° C. for 14 hrs. The dried material was calcined at 575° C. for 4 hours with a ramp rate of 2° C./min in presence of zero air in muffle furnace. Final yield of the dry powder was about 6.2 g.

### Example 7

Preparation of Beta Zeolite Modified with Hexamethyldisilazane

About 7.0 g dried beta zeolite powder in ammonium form was introduced into 250 ml four-neck round bottom flask equipped with addition funnel, reflux condenser, vacuum adaptor, thermometer and magnetic bar. The powder was slowly heated upto 140° C. and evacuated by vacuum up to 10 mbar while stirring. After 4 hours it was cooled down to 50° C. under stirring and then vacuum arrested.

A solution of 2.7 ml of hexamethyldisilazane and 50 ml anhydrous toluene was added in addition funnel. The mixture was carefully added in the RB flask at 50° C. while stirring. After addition of entire quantity, nitrogen gas was introduced into the flask and was heated slowly to reflux temperature under stirring and N₂ gas atmosphere. After 4 hours the flask was cooled down to 40° C. and nitrogen flow was stopped.

The solid was filtered and washed with anhydrous toluene and finally the filtered cake was rinsed with 30 ml of absolute ethanol and dried at 100° C. for 14 hrs. The dried material was calcined at 575° C. for 4 hours with a ramp rate of 2° C./min in presence of zero air in muffle furnace. Final yield of the dry powder was about 6.2 g.

### Example 8

Alkylation of benzene with Ethylene using Various Modified Beta Zeolites

In all cases the catalyst compositions referred earlier in examples 1-7 were mixed thoroughly with suitable support (mostly alumina) in 2:3 ratio and the mixture was pressed at 10 ton pressure to make pellets. The pressed catalyst compositions were crushed and sieved to get the fraction containing particles from 0.5 to 1.00 mm particles for further testing.

0.9 grams of catalyst sample (particle size 0.5-1 0 mm) from each of the combinations (according to examples 1-5) and for comparison of unmodified beta zeolite were loaded in a down flow fixed bed micro-catalytic reactor and pre-treated at 150° C. in a flow of dry nitrogen overnight prior to carrying out reaction.

After the pre-treatment, benzene was fed to the reactor with a syringe pump at 1.5 ml/min to build-up the operating pressure of 35 barg. The temperature of the catalyst bed was then slowly raised from 150 to 220° C. before ethylene flow started. Ethylene flow was set at 5.76 SLPH (Standard litre per hour).

Liquid product samples were taken at regular intervals and analyzed by Gas Chromatography. The unreacted ethylene was measured by wet gas flow meter.

### Conversion:

An indication of the activity of the catalyst was determined by the extent of conversion of the alkylating agent ethylene. The basic equation used was:

Conversion %=Moles of Etyleneᵢₙ−moles of Ethyleneₒᵤₜ/moles of Ethyleneᵢₙ*100/1.

Initial ethylene conversion was close to 99 mole-% and as the reaction proceeds the catalyst gets deactivated. However, in general, silica-modified catalysts showed better resistance to deactivation than unmodified catalysts. The results obtained with different catalysts at reaction temperature of 220° C. are shown in Table 1.

TABLE 1

Ethylene
conversion (mole-%)

| | | 6 hrs | 24 hrs |
|---|---|---|---|
| Example | Zeolites modification | on-stream | on-stream |
| 1 | No modifications | 67.1 | 37.7 |
| 2 | Tetraethoxysilane | 63.2 | 39.7 |
| 3 | Hexamethyldisiloxane | 55.5 | 3.4 |
| 4 | Dichlorodimethylsilane | 87.1 | 72.3 |
| 5 | Methyltrimethoxysilane | 86.6 | 53.1 |
| 6 | Ethoxytrimethylsilane | 83.8 | 57.4 |
| 7 | Hexamethyldisilazane | 96.6 | 80.3 |

These experiments clearly show that deactivation of the catalyst is occurring at a much slower rate (except in case of hexamethyldisiloxane treated catalyst, example 3) in case of silica-modified beta zeolite catalysts than in case of unmodified catalyst (example 1). Thus, according to the present invention silica-modification of the beta zeolite catalyst imparts resistance to deactivation as shown by higher ethylene conversion during the experiments.

### Example 9

Alkylation of Benzene with Ethylene in Presence of Thiophene as Impurity

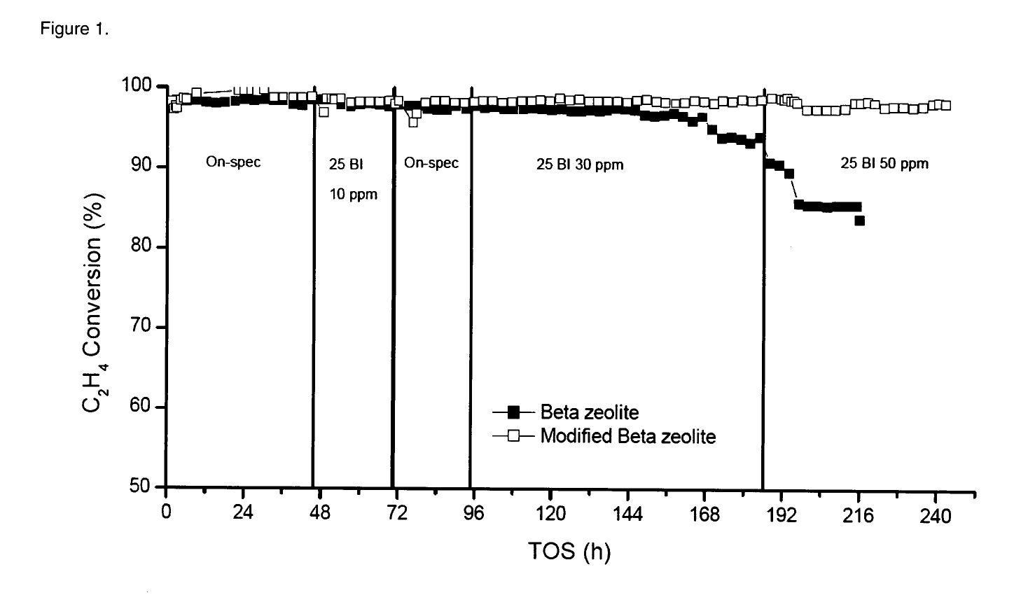

The experimental procedure is similar to experiment 8, only various quantities of thiophene were added to benzene feed as sulphur impurity. Different quantities of hex-1-ene were added as unsaturated hydrocarbon to get the desired bromine index values. The result obtained with beta zeolites modified with hexamethyl-disilazane (example 7) and unmodified beta zeolites are given in FIG. 1. Initially on-spec benzene (bromine index 25 & 0.75 ppm thiophene) was fed to the reactor for 45 hours and then the feed was changed to bromine index 25 and 10 ppm thiophene. After another 24 hours again on-spec benzene was fed for 24 hours and then a feed of bromine index 25 and 30 ppm thiophene was introduced. The initial high ethylene conversion values (99%) for the unmodified beta zeolite start falling from 120 hrs time-on-stream after introduction of this feed. With the introduction of a feed of bromine index 25 and 50 ppm thiophene the ethylene conversion dropped further rapidly. On the other hand the modified beta zeolite performed well with any drop in ethylene conversion in the whole study.

### Example 10 (Comparative)

Preparation of Beta Zeolite Modified with Hexamethyldisilazane while Pores are not filled with Templating Agent during Organosilicon Treatment

9.0 g of beta zeolite powder in ammonium form (with template) was calcined at 550° C. for 4 hours with heating rate of 2° C./min in presence of zero air.

About 7.0 g of this calcined beta zeolite powder was introduced into 250 ml four-neck round bottom flask equipped with addition funnel, reflux condenser, vacuum adaptor, thermometer and magnetic bar. The powder was slowly heated up to 140° C. and evacuated by vacuum up to 10 mbar while stirring. After 4-½ hours it was cooled down to 50° C. under stirring and then vacuum arrested.

A solution of 2.7 ml of hexamethyldisilazane and 50 ml anhydrous toluene was added in addition funnel. The mixture was carefully added in the RB flask at 50° C. while stirring. After addition of entire quantity, nitrogen gas was introduced into the flask and was heated slowly to reflux temperature under stirring and N₂ gas atmosphere. After 4 hours the flask was cooled down to 40° C. and nitrogen flow was stopped.

The solid was filtered and washed with anhydrous toluene and finally the filtered cake was rinsed with 30 ml of absolute ethanol and dried at 100° C. for 14 hrs. The dried material was calcined at 575° C. for 4 hours with a ramp rate of 2° C./min in presence of zero air in muffle furnace. Final yield of the dry powder was about 6.0 g.

TABLE 2

Ethylene conversion (%)

| | Example 7 | Example 10 (comparative) |
|---|---|---|
| TOS (h) | (pores filled) | (pores empty) |
| 3 | 98.47 | 98.37 |
| 6 | 98.43 | 98.29 |
| 9 | 98.54 | 97.80 |
| 12 | 98.43 | 97.64 |
| 15 | 98.58 | 97.39 |
| 20 | 98.48 | 84.22 |
| 25.5 | 98.46 | 68.17 |
| 30 | 98.48 | 47.58 |
| 35 | 98.44 | 35.65 |
| 40 | 98.41 | 20.35 |
| 45 | 98.35 | 13.25 |
| 48 | 98.32 | 7.62 |

The table above shows that after 15 hours of reaction there is a sharp deactivation observed with catalysts prepared from calcined beta zeolite vis-a-vis uncalcined (pores filled with template) beta zeolite using hexamethyldisilazane as silylating agent.

1. A method for preparing a catalyst composition comprising:
(a) contacting an aluminosilicate zeolite having its pores filled with templating agent with an organic silicon compound to deposit said organic silicon compound on the surface of the zeolite to provide an organosilicon treated catalyst precursor; and
(b) calcining the organosilicon treated catalyst precursor under conditions sufficient to remove the templating agent from the zeolite;
wherein the organic silicon compound is selected from the group consisting of:
(I) alkyldisilazane having the general formula 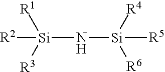
wherein R¹, R², R³, R⁴, R⁵ and R⁶ are alkyl groups independently selected from the group consisting of methyl; ethyl; propyl; butyl; pentyl; hexyl; heptyl; octyl; nonyl; and decyl;
(II) alkylalkoxysilane having the general formula, either of: 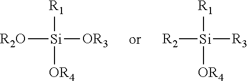
wherein R₁, R₂, R₃ and R₄ are alkyl groups independently selected from the group consisting of methyl; ethyl; propyl; and butyl; and
(III) haloalkylsilane having the general formula 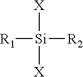
wherein R₁ and R₂ are alkyl groups independently selected from the group consisting of methyl; ethyl; propyl; and butyl and wherein X is an halogen element selected from the group consisting of fluorine (F), chlorine (Cl), bromine (Br) and iodine (I).

2. The method according to claim 1, wherein the alkyl disilazane is selected from the group consisting of hexamethyldisilazane and hexaethyldisilazane.

3. The method according to claim 1, wherein the alkoxy silane is selected from the group consisting of methoxytrimethylsilane, ethoxytrimethylsilane, propoxytrimethylsilane, methyltrimethoxysilane, methyltriethoxysilane, ethyltrimethoxysilane, ethyltriethoxysilane, propyltrimethoxysilane and propyltriethoxysilane.

4. The method according to claim 1, wherein the halo alkyl silane is selected from the group consisting of dichlorodimethylsilane and dichlorodiethylsilane.

5. The method according to claim 1, wherein the zeolite is a large pore size aluminosilicate zeolite.

6. The method according to claim 1, wherein the zeolite is calcined in step (b) at a temperature of 450-600° C. for 3-8 hrs in an oxygen comprising atmosphere.

7. The method according to claim 1, wherein the zeolite is washed in organic solvent before the calcination step (b) is performed.

8. A catalyst composition obtainable by the method according to claim 1.

9. A process for alkylation of an aromatic hydrocarbon comprising contacting the catalyst composition according to claim 8 with a feed stream comprising said aromatic hydrocarbon and an alkylating agent under aromatic alkylation conditions.

10. The process according to claim 9, wherein the aromatic hydrocarbon is benzene and the alkylating agent is ethylene.

11. The process according to claim 10, wherein the benzene:ethylene molar ratio is 3-6:1.

12. The process according to claim 9, wherein the aromatic alkylation is performed in the liquid phase.

13. The process according to claim 9, wherein the aromatic alkylation is performed at a temperature of 150-250° C., a pressure of 5-40 barg, a weight hourly space velocity of 0.1-10.

14. The process according to claim 9, wherein the aromatic hydrocarbon is benzene and the alkylating agent is ethylene; wherein the benzene:ethylene molar ratio is 3-6:1; and wherein the aromatic alkylation is performed in the liquid phase.

15. The process according to claim 14, wherein the aromatic alkylation is performed at a temperature of 150-250° C., a pressure of 5-40 barg, a weight hourly space velocity of 0.1-10.

16. A method for preparing a catalyst composition comprising:
(a) contacting an aluminosilicate zeolite having its pores filled with templating agent with an organic silicon compound to deposit said organic silicon compound on the surface of the zeolite to provide an organosilicon treated catalyst precursor; and
(b) calcining the organosilicon treated catalyst precursor at a temperature of 450-600° C. for 3-8 hrs in an oxygen comprising atmosphere;
wherein the zeolite is washed in organic solvent before the calcination step (b) is performed;
wherein the organic silicon compound is selected from the group consisting of:
(I) alkyldisilazane having the general formula 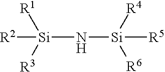
wherein R¹, R², R³, R⁴, R⁵ and R⁶ are alkyl groups independently selected from the group consisting of methyl; ethyl; propyl; butyl; pentyl; hexyl; heptyl; octyl; nonyl; and decyl;
(II) alkylalkoxysilane having the general formula, either of: 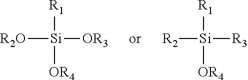
wherein R₁, R₂, R₃ and R₄ are alkyl groups independently selected from the group consisting of methyl; ethyl; propyl; and butyl; and
(III) haloalkylsilane having the general formula 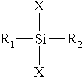
wherein R₁ and R₂ are alkyl groups independently selected from the group consisting of methyl; ethyl; propyl; and butyl and wherein X is an halogen element selected from the group consisting of fluorine (F), chlorine (Cl), bromine (Br) and iodine (I).
